(19) United States
(12) Patent Application Publication
(10) Pub. No.: US 2015/0050620 A1
(43) Pub. Date: Feb. 19, 2015

(54) PREVENTING OR TREATING PERIODONTAL DISEASE

(71) Applicant: KLOX Technologies Inc., Laval (CA)

(72) Inventors: Remigio Piergallini, Grottammare Ascoli Piceno (IT); Nikolaos Loupis, Athens (GR); Shipra Rastogi, Montreal (CA)

(21) Appl. No.: 14/390,015

(22) PCT Filed: Apr. 2, 2013
(86) PCT No.: PCT/CA2013/000314

§ 371 Date: Oct. 1, 2014

Related U.S. Application Data
(63) Continuation-in-part of application No. 13/437,553, filed on Apr. 2, 2012.

(51) Int. Cl. A61C 19/06; A61K 33/40; A61K 45/06; A61K 31/65; A61K 31/43; A61K 31/7052; A61K 6/00; A61K 33/42

(52) U.S. Cl. CPC A61C 19/063; A61K 6/0067; A61K 33/40; A61K 33/42; A61K 31/65; A61K 31/43; A61K 31/7052; A61K 45/06; A61K 6/007
USPC 433/216

(57) ABSTRACT

The disclosure relates to a method for treating or preventing periodontal disease, such as gingivitis or periodontitis. The method may comprise providing an antimicrobial composition disposed within a reservoir, the reservoir being fluidly connected to an exterior surface of an applicator having a frictional stress value sufficient to cause mechanical displacement of a biofilm present on an oral cavity surface; dispensing said antimicrobial composition through said applicator onto said exterior surface of said applicator; applying the antimicrobial composition to the oral cavity surface; and rubbing said applicator onto said oral cavity surface and exerting pressure towards said oral cavity surface.

### FIELD OF THE DISCLOSURE

The disclosure relates generally to preventing or treating periodontal disease, specifically but not exclusively to devices and antimicrobial compositions in methods and uses for preventing or treating periodontal disease.

### BACKGROUND OF THE DISCLOSURE

Periodontal diseases, such as gingivitis and periodontitis, can lead to tooth loss if left untreated. Treatments often require visits to a dental office. Therefore, a need exists for treatments that can be used on a more routine basis outside of a dental office.

### SUMMARY OF THE DISCLOSURE

Disclosed herein is a device and method for applying a brightening or antimicrobial composition to a tooth and/or gums for preventing or treating periodontal disease. The device is portable and can be used outside of a dental office. Preferably, the device applies a certain frictional stress upon a tooth and/or gums. More preferably, the device applies a frictional stress value that allows mechanical displacement of a biofilm present on the surface of a tooth. Even more preferably, the device applies a frictional stress value that allows mechanical displacement of a pellicle layer present on the surface of a tooth, but does not induce damage to the tooth enamel. Also disclosed herein are teeth brightening and antimicrobial compositions for use in conjunction with the device. In certain embodiments, the composition of the disclosure has both teeth whitening and antimicrobial activity. Also disclosed herein are methods for preventing or treating periodontal disease, and uses of the antimicrobial composition and devices for preventing or treating periodontal disease.

In one aspect, there is provided a device comprising a reservoir fluidly connected to an applicator, the applicator having a frictional stress value sufficient to cause mechanical displacement of a biofilm present on a surface of a tooth and/or gum. The applicator may comprise pores having a diameter of 0.1-1000 μm, often but not always in conjunction with the above-indicated frictional stress value. A brightening or antimicrobial composition may be disposed in the reservoir. In some embodiments, the brightening or antimicrobial composition is present in an amount ranging from 2-6 ml. The device may comprise an activator configured to dispense the tooth brightening or antimicrobial composition from the reservoir onto an exterior surface of the applicator.

In some embodiments, the exterior surface of the applicator has a surface area of up to 4-100 mm².

In some embodiments, the applicator comprises a sponge. In some embodiments, the applicator comprises a silicone tip. In some embodiments, the applicator comprises a sponge and the sponge may include pores of sufficient diameter and connectivity for the hydroxyapatite to be transported through the sponge onto an exterior surface of the sponge.

In some embodiments, the brightening or antimicrobial composition comprises a source of peroxide. In some embodiments, the brightening or antimicrobial composition includes hydroxyapatite, such as where the hydroxyapatite has a particle size ranging from 10 to 200 nm.

In another aspect, there is provided a method for teeth brightening. The method may comprise the steps of a) providing a brightening composition disposed within a cylindrical reservoir, the cylindrical reservoir being fluidly connected to an exterior surface of an applicator having a frictional stress value sufficient to cause mechanical displacement of a biofilm present on a surface of the tooth, or comprising pores having a diameter of 0.1-1000 μm, or both, b) dispensing the brightening composition through the applicator onto the exterior surface of the applicator, and c) applying the brightening composition onto a tooth.

In some embodiments, the brightening or antimicrobial composition is applied manually by rubbing the applicator onto a tooth and exerting pressure towards the tooth.

In a further aspect, the disclosure relates to a method for treating or preventing periodontal disease, such as gingivitis or periodontitis. The method may comprise the steps of a) providing a composition disposed within a reservoir, the reservoir being fluidly connected to an exterior surface of an applicator having a frictional stress value sufficient to cause mechanical displacement of a biofilm present on an oral cavity surface, or comprising pores having a diameter of 0.1-1000 μm, or both, b) dispensing the composition through the applicator onto the exterior surface of the applicator, and c) applying the composition to the oral cavity surface. In some embodiments, the oral cavity surface is a tooth and/or gums.

In certain embodiments, the method for treating or preventing periodontal disease, such as gingivitis or periodontitis, comprises applying a composition comprising peroxide such as hydrogen peroxide and/or hydroxyapatite. The composition may comprise an antimicrobial agent such as an antibiotic or antiviral agent. Exemplary antibiotic agents that may be included in the composition include tetracycline, doxycycline, minocycline, amoxicillin and azithromycin.

In the method for treating gingivitis or periodontitis, the composition may be applied to the oral cavity surface, such as teeth and/or gums, at least once daily or at least once weekly. In particular embodiments, the composition is applied to the gums and/or the tooth/gum junction.

From another aspect, there is provided a method for preventing or treating periodontal disease, comprising: providing an antimicrobial composition disposed within a reservoir, the reservoir being fluidly connected to an exterior surface of an applicator having a frictional stress value sufficient to cause mechanical displacement of a biofilm present on an oral cavity surface; dispensing said antimicrobial composition through said applicator onto said exterior surface of said applicator; applying the antimicrobial composition to the oral cavity surface; and rubbing said applicator onto said oral cavity surface and exerting pressure towards said oral cavity surface. In certain embodiments, rubbing said applicator onto said oral cavity surface and exerting pressure towards said oral cavity surface occurs at the same time as, before, or after applying the antimicrobial composition to the oral cavity surface. The method may include a further step of applying new antimicrobial composition to the oral cavity surface following rubbing said applicator onto said oral cavity surface and exerting pressure towards said oral cavity surface. The application of the antimicrobial composition may be as a flow of composition from the reservoir to the exterior surface of the reservoir. The flow may be continuous or otherwise.

From another aspect, there is provided a method for preventing or treating periodontal disease, comprising: mechanically displacing a biofilm on an oral cavity surface; and applying an antimicrobial composition to the oral cavity surface. In certain embodiments, applying the antimicrobial composition comprises contacting the oral cavity surface with an exterior surface of an applicator fluidly connected to a reservoir containing the antimicrobial composition. In certain embodiments, mechanically displacing the biofilm comprises rubbing said oral cavity surface and exerting pressure towards said oral cavity surface with the external surface of the applicator, the external surface of the applicator having a frictional stress value sufficient to cause mechanical displacement of the biofilm on the oral cavity surface. In certain embodiments, the antimicrobial composition is applied to the oral cavity surface at the same time as, before, or after mechanically displacing the biofilm on the oral cavity surface. In certain embodiments, new antimicrobial composition is applied to the oral cavity surface after displacing the biofilm.

In certain embodiments of the aspects of the methods above, the antimicrobial composition may be broken down by enzymes on the oral cavity surface or in the saliva. Therefore, providing fresh antimicrobial composition to the oral cavity surface, which may be a continuous or interrupted supply, can provide continued antimicrobial effect which may result in a faster treatment or more effective prevention of the periodontal disease.

In certain embodiments of the aspects of the methods above, the oral cavity surface is a gum surface, a tooth surface, or both gum and tooth surfaces. The gum or tooth surfaces can include periodontal pockets, in which case the method can further comprise pushing the antimicrobial composition into the periodontal pockets.

In certain embodiments of the aspects of the methods above, the antimicrobial composition comprises a peroxide source which can be hydrogen peroxide or urea peroxide.

In certain embodiments of the aspects of the methods above, the antimicrobial composition includes hydroxyapatite which may have a particle size ranging from 10-200 nm. In certain embodiments, the antimicrobial composition comprises an antibiotic agent which may be selected from tetracycline, doxycycline, minocycline, amoxicillin and azithromycin. The antimicrobial composition may comprises one or more of: fluoride, triclosan, sodium bicarbonate, sweetening agent, detergent, chlorhexidine, cetylpyridinium, stannous fluoride and an amine fluoride.

In certain embodiments of the aspects of the methods above, the antimicrobial composition is applied to the oral cavity surface at least once daily, or at least once weekly.

In certain embodiments of the aspects of the methods above, a contact area between the exterior surface of the applicator and the oral cavity surface is about 0.25-400 mm², about 4-100 mm² or about 9-25 mm². The exterior surface of the applicator may have a frictional stress value greater than about 0.001 N/mm². The applicator may comprise pores having a diameter of 0.1-1000 μm. The exterior surface of the applicator may be made of any suitable material or have any suitable texture for mechanically removing biofilm. For example, the applicator may comprise a sponge or silicone. The exterior surface of the applicator may comprise bristles.

From a yet further aspect, there is provided use of an antimicrobial composition for preventing or treating periodontal disease, wherein the antimicrobial composition is disposed within a reservoir fluidly connected to an exterior surface of an applicator for dispensing the antimicrobial composition through the applicator onto the exterior surface of the applicator for applying the antimicrobial composition to the oral cavity surface, said exterior surface of the applicator having a frictional stress value sufficient to cause mechanical displacement of the biofilm when the applicator exterior surface is rubbed against the oral cavity surface and pressure is exerted towards said oral cavity surface.

From another aspect, there is provided use of an antimicrobial composition on an oral cavity surface with mechanical displacement of a biofilm on the oral cavity surface, for preventing or treating periodontal disease. There is also provided use of an antimicrobial composition on an oral cavity surface having a disrupted biofilm thereon, for preventing or treating periodontal disease. There is also provided use of an applicator for treating or preventing periodontal disease, wherein the applicator has an external surface for applying an antimicrobial composition to an oral cavity surface applicator, the external surface having a frictional stress value sufficient to cause mechanical displacement or disruption of the biofilm present on the oral surface cavity. In certain embodiments, the antimicrobial composition is disposed within a reservoir fluidly connected to an exterior surface of an applicator for contacting the oral cavity surface to apply the antimicrobial composition to the oral cavity surface, said applicator having a frictional stress value sufficient to cause mechanical displacement or disruption of the biofilm present on the oral surface cavity.

In certain embodiments of the above uses, the applicator and reservoir are arranged to deliver the antimicrobial composition onto the exterior surface of the applicator at the same time as, before, or after, mechanical displacement of the biofilm, to prevent or treat periodontal disease. In certain embodiments of the above uses, the oral cavity surface is a gum surface, a tooth surface, or both gum and tooth surfaces.

In certain embodiments of the above uses, the antimicrobial composition comprises a peroxide source, such as urea peroxide. In certain embodiments of the above uses, the antimicrobial composition includes hydroxyapatite, which may have a particle size ranging from 10-200 nm. The antimicrobial composition may comprise one or more of fluoride, triclosan, sodium bicarbonate, sweetening agent, detergent, chlorhexidine, cetylpyridinium, stannous fluoride and an amine fluoride. The antimicrobial composition may comprise an antimicrobial agent which may be an antibiotic agent, for example, tetracycline, doxycycline, minocycline, amoxicillin and azithromycin, or an antiviral agent.

In certain embodiments of the above uses, a contact area between the exterior surface of the applicator and the oral cavity surface is about 0.25-400 mm², about 4-100 mm² or about 9-25 mm². The applicator may have a frictional stress value greater than about 0.001 N/mm². The applicator may comprise pores having a diameter of 0.1-1000 μm. The exterior surface of the applicator may be made of any suitable material or have any suitable texture for mechanically removing biofilm. For example, the applicator may comprise a sponge or silicone. The exterior surface of the applicator may comprise bristles.

In certain embodiments of the above aspects, the antimicrobial composition comprises urea peroxide due to its ability to generate reactive oxygen species which can prevent or treat periodontal disease, and due to its lack of side effects. In particular, urea peroxide is less easily enzymatically degraded compared to hydrogen peroxide, and is better tolerated by the mucosa of the oral cavity. Furthermore, urea peroxide does not stain teeth, does not alter taste, and does not induce sensitivity, which can occur with other antimicrobial agents such as chlorhexidine.

### BRIEF DESCRIPTION OF THE DRAWINGS

Further aspects and advantages of the present disclosure will become better understood with reference to the description in association with the following in which:

FIG. 1 shows a device for applying a brightening or antimicrobial composition to a tooth and/or gums.

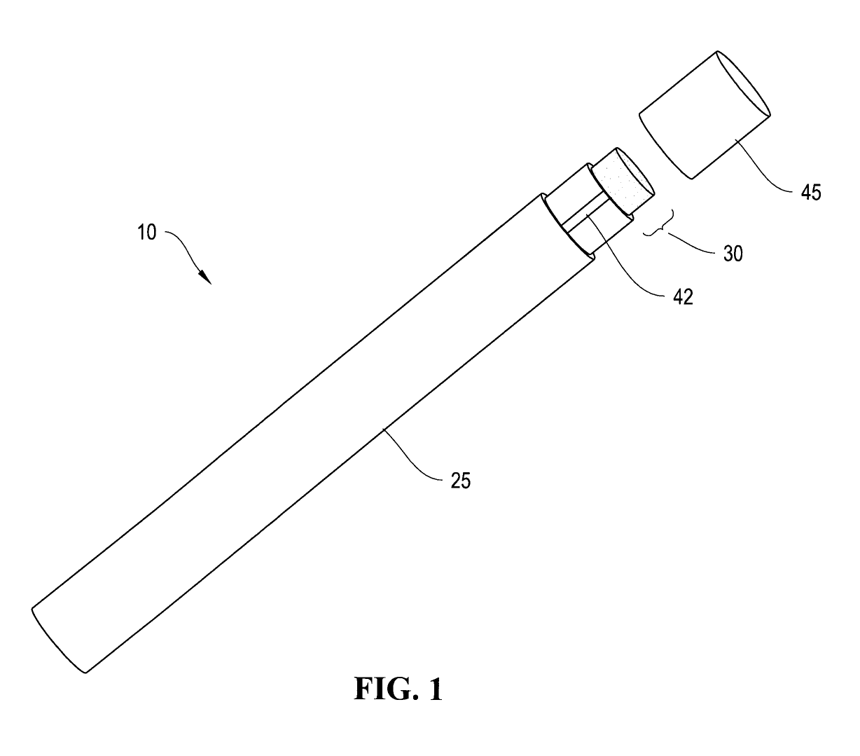

FIG. 2 shows a device for applying a brightening or an antimicrobial composition to a tooth and/or gums, wherein the device has an applicator comprising a sponge.

FIG. 3 depicts the antibacterial activity of the composition in a disc diffusion assay. The antibacterial activity of the composition is compared to peroxide positive controls as well as saline negative control.

FIG. 4 depicts the inhibition of bacterial growth around the disc in the assay of FIG. 3.

### DETAILED DESCRIPTION

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. In the following description, the same numerical references refer to similar elements. In the drawings, like reference characters designate like or similar parts.

Devices and Methods

Described herein is a device 10 and method for applying a brightening or antimicrobial composition to a tooth and/or gums. Preferred devices comprise a reservoir 25 fluidly connected to an applicator 30. Preferably, the applicator is optimized with regard to its mechanical performance so as to induce a certain frictional stress upon a tooth and/or gums. An advantageous choice of frictional stress allows effective tooth brightening or prevention or treatment of periodontal disease, such as gingivitis or periodontitis, but without damaging the enamel or another portion of the tooth or gums. However, the frictional stress should be sufficient to mechanically displace a biofilm and/or pellicle layer (partially or completely) from the surface of a tooth and/or gums. “Pellicle” as used herein is a layer of salivary glycoproteins that adhere to the surface of a tooth and/or gums. “Biofilm” as used herein is a substance that adheres to the surface of the tooth and/or gums or the pellicle layer, and may include additional components, for example substances excreted from bacteria. For example, a biofilm may include a sessile community of cells that may be microbioally derived and that are attached to a substrate or to each other. These adherent cells are often embedded in a matrix of extracellular polymeric substances that they have produced, and that exhibit an altered phenotype with respect to growth rate and gene transcription.

The device 10 places an applicator 30 in contact with the surface of a tooth and/or gums and delivers a tooth brightening or antimicrobial composition through the applicator 30 onto the surface of a tooth and/or gums.

The device 10 preferably has a reservoir 25 for storing the brightening or antimicrobial composition and an applicator 30 that is fluidly connected to the reservoir 25 via a feeder 42. The device 10 may be portable and in the shape of a pencil, pen or liquid stick. In one embodiment, the device 10 includes more than one applicator 30 that may be removably engaged with the device 10. In an embodiment wherein the device 10 is a shaped like a pen or a pencil, the applicator 30 may be retractable. The brightening or antimicrobial composition described herein may be housed directly within the reservoir 25 in the device 10 or may be supplied in a removable cartridge (not shown) within the reservoir 25 that may be replaced or refilled. The device may also comprise a cap 45.

It is believed that the performance of the applicator 30 with regards to tooth brightening or antimicrobial activity is enhanced by the friction or abrasion induced by the applicator 30 upon a tooth and/or gums. The friction or abrasion induced by the applicator 30 upon a tooth and/or gums can be defined in terms of frictional stress. The frictional stress value can be defined as the force exerted upon a reference surface per unit area of real contact, which is expressed as σ=T/A=μN/A. In this equation, T is the tangential force, A the area of contact, μ the coefficient of friction, and N the vertical force.

Without being bound by theory, it is believed that the frictional stress of an applicator 30 is of particular importance, as it indicates the efficiency with which the mechanical energy provided by the user is transferred to the surface of a tooth and/or gums. The frictional stress can cause mechanical displacement of biofilm. When an applicator has a low frictional stress value, the energy supplied by the user is dissipated in other ways, for example, through the applicator itself, resulting in the applicator deforming.

Frictional stress values greater than 0.001 N mm ⁻² are advantageous. More preferably, the frictional stress values are from 0.001-0.01 N mm⁻², 0.01-0.1 N mm⁻², 0.1 to 0.2 N mm⁻², 0.2-0.3 N mm⁻², 0.3-0.4 N mm⁻², 0.4-0.5 N mm⁻², 0.5-0.6 N mm⁻², 0.6-0.7 N mm⁻², 0.7-0.8 N mm⁻², 0.8-0.9 N mm⁻², or 0.9-1 N mm⁻². In certain embodiments, the frictional stress values may be from 1-1.5 N mm⁻², 1.5-2 N mm⁻², or even from 2-2.5 or 2.5-3 N mm⁻².

The frictional stress value of an applicator 30 may be measured using methods known in the art. One example uses a Plint dual axis reciprocating rig (such as model TE75R, MRPRA RUBBER CONSULTANTS). The device 10 is clamped to the load arm of the reciprocating rig and the angle of the device relative to the reference surface is adapted to maximize the contact area of an exterior surface of the applicator 30. The clamping arrangement should be set to provide a consumer realistic normal force, N, on the applicator 30 of about 3 N. The coefficient of friction is then measured between the applicator 30 and a reference surface that is similar to the surface of a tooth and/or gums. The applicator 30 is measured wet using a brightening or antimicrobial composition as given in Example 1. The coefficient of friction is measured over the central 10 mm of four traverses of 20 mm in both the forward and reverse direction at a speed of 1 mm s⁻¹ and an average value calculated. Measurements with the applicator 30 in final measuring position are repeated three times to check reproducibility.

In some embodiments, the applicator (e.g., 30) comprises a sponge 40. The sponge 40 includes pores 41 that are fluidly connected to a feeder (e.g., 42) which receives the brightening or antimicrobial composition from reservoir (e.g., 25). Preferably, the sponge 40 is comprised of pores 41 having a mean diameter of about 0.1-1000 μm. More preferably, the pores have a mean diameter of about 0.1-100 μm, 100-200 μm, 200-300 μm, 300-500 μm, 500-750 μm or 750-1000 μm.

The sponge 40 may be made of synthetic or man-made or natural materials such as felt, foam, polyethylene, nylon, silicone, etc. Preferably, the sponge 40 is made of a material resistant to peroxide-induced corrosion.

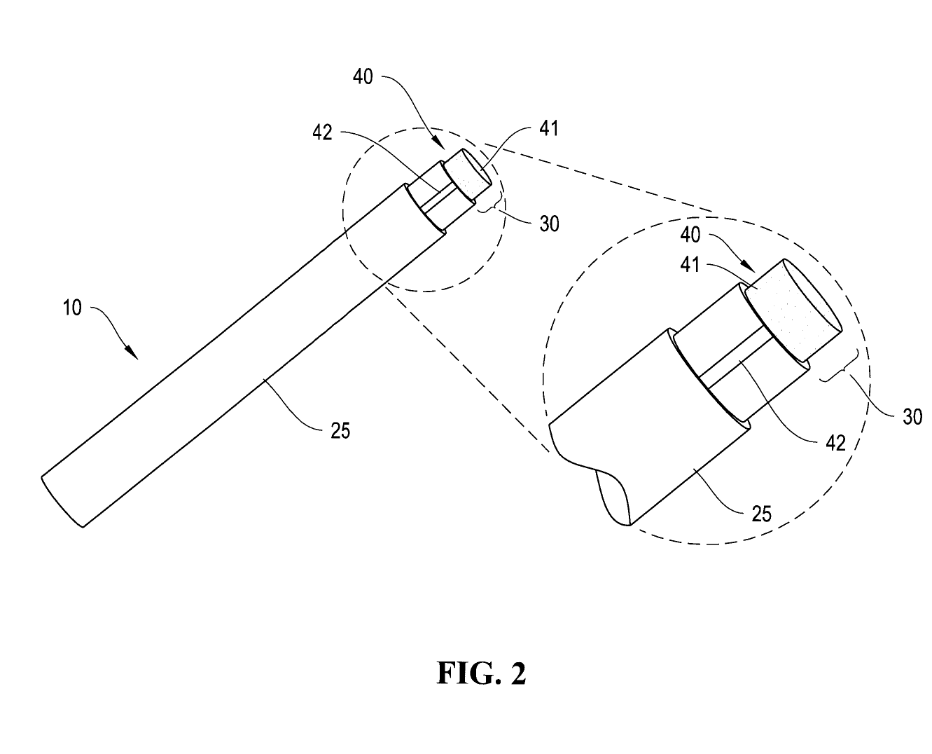

In some embodiments, the applicator comprises a sponge 40 made of nylon such as flocked nylon. FIG. 2 illustrates an exemplary embodiment in which the applicator comprises a sponge 40. As shown in FIG. 2, the sponge 40 is optimized with regard to having pores 41 of sufficient diameter and connectivity for particles (e.g., hydroxyapatite particles, as described below) in the brightening or antimicrobial composition to be transported through the pores 41 onto an exterior surface of the applicator 30. BrightOne (www.blancone.it) sold by International Dental Supply includes a sponge having the above described characteristics.

The contact area between the exterior surface of the applicator 30 and a surface of a tooth and/or gums preferably is from 0.25-400 mm², 4-100 mm², or 9-25 mm². Such a contact area ensures optimal mechanical removal of biofilm and allows for efficient application of the brightening or antimicrobial composition onto the surface of a tooth and/or gums.

Measurements of the contact area of the exterior surface of the applicator 30 can be carried out with a dry applicator 30. The dry applicator 30 is wetted by pressing it against a pad containing a brightening or antimicrobial composition and then clamping the device 10 to the load arm of a plint dual axis reciprocating rig (such as model TE75R, MRPRA RUBBER CONSULTANTS). A mark on a contact surface that is representative of the contact area of the exterior surface of the applicator 30 is obtained by controlled lowering and rising of the plint load arm towards and away from the reference surface. The angle of the device relative to the reference surface is adapted to maximize the contact area between the applicator 30 and the reference surface. The contact time should be approximately 1 s while a normal load of about 3 N should be applied on the application device. The contact area can then be calculated from the mean length and width of the mark determined using a magnifying lens with a graticule. Measurements with the applicator 30 in final measuring position are repeated three times to check reproducibility.

The device 10 may dispense the brightening or antimicrobial composition from the reservoir 25 onto an exterior surface of the applicator 30 through the feeder 42 via capillary action, such as in a flow through pen, or by exerting pressure on the applicator 30 by pushing the device 10 onto a surface of a tooth and/or gums, or via an activator, such as a mechanical piston with a click mechanism, a twist button and ratchet mechanism, or a push button mechanism, or through a vacuum method of ejection, or through other mechanical means that transfer the composition from the reservoir 25 to an oral cavity surface in need of treatment. The activator may be positioned on a first end or side wall of the device 10.

In certain embodiments, the device has an activator comprising a push button. With the push button activator, the user pushes the button located on a first end or side wall of the device 10, which causes the transfer of the composition from the reservoir 25 through the feeder 42 and onto the exterior surface of the applicator 30. More preferably, push button activator has an arrangement that allows partitioning of the brightening composition. This can be achieved, for example, via a catch arresting mechanism connected to the push button activator. From the sound of the catch upon actuating the push button activator, the user is able to recognize that a single dose of the brightening or antimicrobial composition has been dispensed onto the exterior surface of the applicator 30.

Once the composition is positioned onto the exterior surface of the applicator 30, a user applies the composition to a tooth and/or gum surface by manually rubbing the applicator 30 onto the tooth and/or gums and exerting pressure towards the tooth and/or gums. Preferably, manually rubbing the applicator 30 onto the tooth and/or gums and exerting pressure towards the tooth and/or gums causes mechanical displacement of a biofilm from the tooth and/or gum surface.

A set of instructions can be provided to the user to describe how to apply the composition from the device 10 onto the teeth and/or gums.

In certain embodiments, the reservoir 25 is made of peroxide-resistant materials. In one embodiment, the reservoir 25 is made from fluoropolymers, polypropylene, polyethylene, or other such polymers that are compatible with the ingredients of the composition of the present disclosure.

Compositions

The devices of the disclosure may be used for delivering a variety of compositions in particular to the teeth and gums. In some embodiments, the devices deliver compositions that brighten teeth and/or have antimicrobial properties. The compositions described herein may be used for the treatment of periodontal disease such as gingivitis or periodontitis. Compositions with antimicrobial activity may also have teeth whitening activity.

The term “preventing” is art-recognized, and when used in relation to a condition, such as a periodontal disease such as periodontitis, is well understood in the art, and includes administration of a composition which reduces the frequency of, or delays the onset of, symptoms of a medical condition in a subject relative to a subject which does not receive the composition. Thus, prevention of periodontitis includes, for example, reducing the inflammation or infection of the ligaments and bones that support the teeth in a population of patients receiving a prophylactic treatment relative to an untreated control population.

The term “prophylactic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition.

“Treating” a condition or disease refers to curing as well as ameliorating at least one symptom of the condition or disease.

The composition used herein is comprised of a gel carrier and at least one brightening agent dispersed throughout the gel carrier. The brightening agent or antimicrobial agent may be dissolved in the gel carrier or simply dispersed homogeneously in the carrier as an insoluble suspended solid particulate.

The composition may comprise a peroxide source. Hydrogen peroxide is a powerful oxidizing agent. Typically, the concentration of hydrogen peroxide in the present composition is from about 0.001-10% by weight of the composition, such as 1-7% or 4-6%. Urea hydrogen peroxide (also known as urea peroxide, carbamide peroxide or percarbamide) may also be used. Typically, the concentration of urea peroxide in the present composition is from about 0.003-30% by weight of the composition, such as about 1-25%, 10-20% or 13-17%. The composition may comprise a bicarbonate salt such as sodium bicarbonate.

The gel carrier may contain any number of ingredients that increase the viscosity of the composition and may be present in an amount ranging from about 35-95%, or 45-70%, or 55-65% by weight of the composition. In certain embodiments, sufficient gel carrier is added to obtain a composition having a viscosity of about 10,000 to 200,000 cps, or about 30,000 to 150,000, or 50,000 to 120,000. The gel carrier may comprise one or more polymers. Preferred polymers are high molecular weight polymers of acrylic acid such as Carbopol®. The gel carrier may also include cellulose derivatives (such as hydroxyethyl cellulose, carboxymethyl cellulose sodium, and methyl cellulose), gums (such as sodium alginate, carrageenan, xanthan gum, tragacanth gum, acacia gum, jellan gum, and native jellan gum), synthetic binders (such as polyvinyl alcohol, carboxyvinyl polymer, polyvinyl pyrrolidone, propylene glycol and polyethylene oxide), natural polyols (such as glycerin, mannitol, sorbitol and maltitol) and inorganic binders (such as silica gel, aluminum silica gel, bee gum, and Laponite). For example, the gel carrier may contain a polymer in combination with a synthetic binder and/or a natural polyol, such as 12-18% synthetic binder (e.g., propylene glycol), 40-50% natural polyol (e.g., glycerin), and 0.5-4% of polymer (e.g., Carbopol).

In some embodiments, the composition comprises flavoring agents. Flavoring agents that are useful include essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole, and synthetic flavors like Evercool, and derivatives of cyclic alpha-keto enamines. Of these, the most commonly employed are the oils of peppermint, spearmint and wintergreen. The flavoring agent is incorporated in the brightening liquid composition of the present disclosure at a concentration of about 0.05 to about 2%, or preferably about 0.1 to about 0.5% by weight of the composition. A sweetening material may also be employed as a complement to the flavoring material. Suitable sweetening agents are water soluble and include sodium saccharin, sodium cyclamate, xylitol, perillartien, D-tryptophan, aspartame, dihydrochalcones and the like.

In some embodiments, the composition comprises a brightening particulate. The brightening particulates may comprise a form of calcium phosphate. The calcium phosphate may have a structure selected from tetracalcium phosphate, amorphous calcium phosphate, alpha-tricalcium phosphate, beta-tricalcium phosphate and hydroxyapatite (Ca₅ (OH)(PO₄)₃). The calcium phosphate may be a substantially aqueous insoluble calcium phosphate and non-crystalline, poorly crystalline or crystalline form such as, for example, crystalline hydroxyapatite. Preferably, the composition includes nanoparticles of hydroxyapatite.

Hydroxyapatite has a similar physical structure as tooth enamel, and thus has a strong affinity to the tooth enamel surfaces, resulting in the hydroxyapatite particulates imparting a “natural” white appearance to the enamel surface. The hydroxylapatite crystals may also cause accumulation of an electrostatic charge, due to the pressure and friction exerted by the sponge, facilitating and amplifying the penetration of ions in the enamel structure. A large amount of the nanoparticles of hydroxyapatite could even percolate in the enamel and facilitate a deposition of calcium and calcium phosphate ions on the enamel. The hydroxyapatite may also decrease the deleterious effects of peroxides and allow remineralization of the enamel. Preferably, hydroxyapatite is present in the composition of the present disclosure at a concentration of about 0.5-5%, or about 1-2% by weight of the composition. In some embodiments, hydroxyapatite particles can comprise aggregates of individual hydroxyapatite particles. For example, such aggregates can have a mean diameter of from about 100 nm to about 1000 nm, and comprise hydroxyapatite particles having a mean diameter of about 10 nm to about 200 nm.

In some embodiments, the composition further comprises one or more of fluoride, triclosan, detergent, clorhexidine, cetylpyridinium, stannous fluoride, and an amine fluoride. Examples of amine fluorides include olaflur (N′-octadecyltrimethylenediamine-N,N,N′-tris(2-ethanol)-dihydrofluoride) and dectaflur (9-octadecenylamine-hydrofluoride). Exemplary detergents include delmopinol, sodium lauryl sulfate (SLS), and cocoamidopropyl betaine (CAPB).

In certain embodiments, the composition further comprises an antimicrobial agent. For example, the antimicrobial agent may be selected from an antiviral agent or an antibiotic agent. Antibiotics include, for example, vancomycin, penicillin, amoxicillin, ampicillin, cefotaxime, ceftriaxone, cefixime, rifampinmetronidazole, doxycycline, tetracycline, minocycline, azithromycin, tacrolimus, cyclosporine, sirolimus, everolimus, ascomycin, erythromycin, clarithromycin, clindamycin, lincomycin, dirithromycin, josamycin, spiramycin, diacetyl-midecamycin, tylosin, roxithromycin, ABT-773, telithromycin, leucomycins, lincosamide, dactinomycin, daunorubicin, doxorubicin, idarubicin, anthracyclines, mitoxantrone, bleomycins, plicamycin, mitomycin and streptomycin.

In some embodiments, the composition comprises a stabilizing agent. The stabilizing agent utilized in the aqueous gel is present in an amount ranging from about 0.01% to about 5% by weight of the aqueous gel. An amount of approximately 1% stabilizing agent is preferred. The stabilizing agent is typically selected from aminocarboxylic acids and salts thereof. Preferred stabilizers are selected from aminocarboxylic acids and alkali and/or alkali earth metal salts thereof. Suitable aminocarboxylic acids include trans-1,2-cyclohexylene dinitrilotetraacetic acid (CDTA), ethylenediamine tetraacetic acid (EDTA), N-(2-hydroxyethyl)ethylenediamine triacetic acid (HEDTA), Nitrilotriacetic acid (NTA), diethylene triamine pentaacetic acid (DTPA), triethylene tetraamine hexaacetic acid (TTHA), and ethyleneglycol bis(2-aminoethylether) tetraacetic acid (GEDTA).

In addition to the aforementioned components, a neutralizing agent may be added to the composition. The inorganic and organic neutralizing agents which may be employed are bases. Suitable bases include alkali metal hydroxides and ammonium hydroxide, carbonates, alkoxides, oxides, peroxides, superoxides, and water soluble organic amines. Amino acids such as β-alanine and lysine can also be used for neutralization and viscosity modification. Preferred bases include sodium hydroxide, potassium hydroxide, ammonium hydroxide, triethanolamine (TEA), aminomethyl propanol (AMP), 2-amino-2-hydroxymethyl-1,3 -propanediol (Tromethamine), tetrahydroxypropyl ethylenediamine, and tris(hydroxymethyl)aminomethane (TRIS). In certain embodiments, the neutralizing agent will be used to obtain a brightening composition having a pH of about 4 to 10, or about 5 to 7, or about 5.5 to 6.5.

The following are non-limiting examples of aspects and embodiments of the present disclosure.

### EXAMPLE 1

An exemplary composition was prepared by mixing the following components.

| Components | % content |
|---|---|
| Propylene Glycol | ≈16% |
| Purified Water | ≈18% |
| Glycerin | ≈45% |
| Carbamide Peroxide | ≈15% |
| Disodium EDTA | ≈1% |
| CARBOPOL 940 | ≈2% |
| Sodium Hydroxide | ≈0.5% |
| Nano-crystals Hydroxylapatite | ≈1.5% |
| Sodium Saccharine | ≈0.1% |
| Mint Flavoring | ≈0.2% |

### EXAMPLE 2

Evaluation of the antibacterial activity of the composition using a disc diffusion assay.

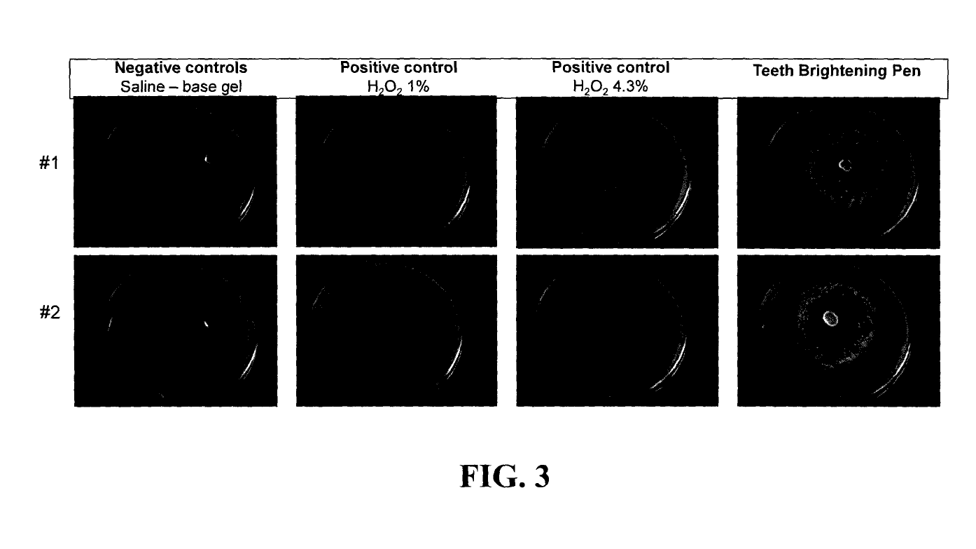

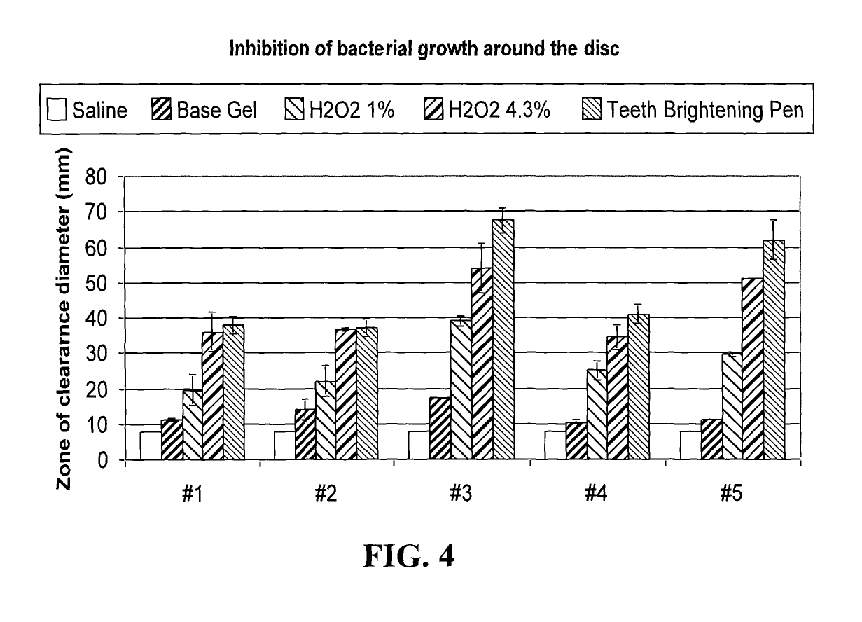

Nutrient medium was inoculated with fresh bacteria from the gum-teeth junction and cultured at 37° C. with agitation. 24 h later 500 μL of bacterial suspension were spread onto agar petri dishes, and sterile nylon discs of 8 mm were placed on the plates. 15 μL of test solutions (saline, 1% H₂O₂ and 4.3% H₂O₂) or 500 μL of the composition of Example 1 were applied on the disc, and the plates were incubated at 37° C. for 24 h. As shown in FIGS. 3 and 4, the composition of Example 1 displays a clear bactericidal activity on bacteria from gum-teeth junction as illustrated by the diameter of bacteria-free zone around the disc.

It should be appreciated that the disclosure is not limited to the particular embodiments described and illustrated herein but includes all modifications and variations falling within the scope of the disclosure as defined in the appended claims.

1. A method for preventing or treating periodontal disease, comprising:
providing an antimicrobial composition disposed within a reservoir, the reservoir being fluidly connected to an exterior surface of an applicator having a frictional stress value sufficient to cause mechanical displacement of a biofilm present on an oral cavity surface; dispensing said antimicrobial composition through said applicator onto said exterior surface of said applicator;
applying the antimicrobial composition to the oral cavity surface; and
rubbing said applicator onto said oral cavity surface and exerting pressure towards said oral cavity surface.

2. The method of claim 1, wherein rubbing said applicator onto said oral cavity surface and exerting pressure towards said oral cavity surface occurs: a) at the same time as; b) before; or c) after applying the antimicrobial composition to the oral cavity surface.

3.-4. (canceled)

5. The method of claim 1, further comprising a step of applying new antimicrobial composition to the oral cavity surface following rubbing said applicator onto said oral cavity surface and exerting pressure towards said oral cavity surface.

6. The method of claim 1, wherein the oral cavity surface is a gum surface, a tooth surface, or both gum and tooth surfaces.

7. The method of claim 6, wherein the gum or tooth surfaces include periodontal pockets, said method further comprising pushing the antimicrobial composition into the periodontal pockets.

8. The method of claim 1, wherein said antimicrobial composition comprises a peroxide source.

9. The method of claim 8, wherein the peroxide source is urea peroxide.

10. The method of claim 1, wherein said antimicrobial composition includes hydroxyapatite.

11. The method of claim 10, wherein the hydroxyapatite has a particle size ranging from 10-200 nm.

12. The method of claim 1, wherein said antimicrobial composition comprises an antimicrobial agent.

13. The method of claim 12, wherein the antimicrobial agent is an antibiotic agent.

14. The method of claim 13, wherein the antibiotic agent is selected from tetracycline, doxycycline, minocycline, amoxicillin and azithromycin.

15. The method of claim 12, wherein the antimicrobial agent is an antiviral agent.

16. The method of claim 1, wherein the antimicrobial composition comprises one or more of: fluoride, triclosan, sodium bicarbonate, sweetening agent, detergent, chlorhexidine, cetylpyridinium, stannous fluoride and an amine fluoride.

17. The method of claim 1, wherein the composition is applied to the oral cavity surface at least once daily.

18. The method of claim 1, wherein the composition is applied to the oral cavity surface at least once weekly.

19. The method of claim 1, wherein a contact area between the exterior surface of the applicator and the oral cavity surface is about 0.25-400 mm², about 4-100 mm² or about 9-25 mm².

20. The method of claim 1, wherein the applicator has a frictional stress value greater than about 0.001 N/mm².

21. The method of claim 1, wherein the applicator comprises pores having a diameter of 0.1-1000 μm.

22. The method of claim 1, wherein the exterior surface of the applicator comprises bristles.

23. A method for preventing or treating periodontal disease, comprising:
mechanically displacing a biofilm on an oral cavity surface; and
applying an antimicrobial composition to the oral cavity surface.

24. The method of claim 23, wherein applying the antimicrobial composition comprises contacting the oral cavity surface with an exterior surface of an applicator fluidly connected to a reservoir containing the antimicrobial composition.

25. The method of claim 24, wherein mechanically displacing the biofilm comprises rubbing said oral cavity surface and exerting pressure towards said oral cavity surface with the external surface of the applicator, the external surface of the applicator having a factional stress value sufficient to cause mechanical displacement of the biofilm on the oral cavity surface.

26. The method of claim 1, wherein the antimicrobial composition is applied to the oral cavity surface at the same time as mechanically displacing the biofilm on the oral cavity surface.

27. The method of claim 23, wherein the antimicrobial composition is applied to the oral cavity surface before mechanically displacing the biofilm on the oral cavity surface.

28. The method of claim 23, wherein the antimicrobial composition is applied to the oral cavity surface after mechanically displacing the biofilm on the oral cavity surface.

29. The method of claim 23, wherein applying said antimicrobial composition to the oral cavity surface comprises supplying a flow of said antimicrobial composition from a reservoir to an applicator which is rubbed on the oral cavity surface.

30. The method of claim 23, wherein the oral cavity surface is a gum surface, a tooth surface, or both gum and tooth surfaces.

31. The method of claim 30, wherein the gum or tooth surfaces includes periodontal pockets, said method further comprising pushing the antimicrobial composition into the periodontal pockets.

32.-83. (canceled)
